(19) United States
(12) Patent Application Publication
(10) Pub. No.: US 2021/0169742 A1
(43) Pub. Date: Jun. 10, 2021

(54) SYSTEM AND METHOD FOR CONTROLLED DISPENSING OF MEDICATION

(71) Applicant: Bradley Paul Willett, Scottsdale, AZ (US)

(72) Inventor: Bradley Paul Willett, Scottsdale, AZ (US)

(21) Appl. No.: 16/708,941

(22) Filed: Dec. 10, 2019

(51) Int. Cl. A61J 7/04; G16H 20/13; A61J 1/03

(52) U.S. Cl. CPC A61J 7/0481; A61J 1/03; G16H 20/13

(57) ABSTRACT

A medication dispensing device and method that forces patient compliance with a health-care-provider-prescribed medication schedule. The device may be filled with medication by a health care provider, but not by the patient. The device may be programmed to limit access to medication with prescribed minimum time intervals between access to doses. These minimum time intervals may be uniform or flexible. The device may unlock to grant a patient access to a dose of medication through a lock. The device may also be programmed to prevent any release of medication after a prescribed period of time has elapsed. The device may include an insert to prevent cross contamination between uses. The device may include an unlocked compartment housing limited amounts of medication to be accessed as needed. The device may be returned to a health care provider to dispose of excess medication.

### CROSS REFERENCE TO RELATED APPLICATIONS

This application claims priority to provisional U.S. Patent Application No. 62/834,322 filed Apr. 15, 2019, the contents of which are hereby incorporated by reference in their entirety.

### BACKGROUND

### 1. Field of the Invention

This invention relates to systems and methods for ensuring patient compliance with medication schedules.

### 2. Description of Related Art

To reduce the complexity and length of the Detailed Description, and to fully establish the state of the art in certain areas of technology, Applicant herein expressly incorporates by reference all the following materials identified in each numbered paragraph below.

1. U.S. Pat. No. 8,744,620 discloses a medication dispensing system with locking compartments; U.S. Pat. No. 9,717,654 discloses a method for refilling electronic pill boxes with pre-loaded packs; U.S. Pat. No. 9,387,154 discloses a pillbox and method for forced patient adherence to medication schedules; U.S. Pat. No. 7,896,192 discloses a device and method for controlling medication scheduling through prescribed minimum times between doses; U.S. Pat. No. 9,842,512 discloses a device and method for tapering prescription drug use in post-operative patients; US Publication No. 20120281889 discloses a safe accessible through fingerprint access; US Publication No. 20080027579 discloses a medication dispenser that provides access to only one dose at a time; and U.S. Pat. No. 4,748,600 discloses a rotating medication dispenser.

Applicant believes that the material incorporated above is “non-essential” in accordance with 37 CFR 1.57, because it is referred to for purposes of indicating the background of the invention or illustrating the state of the art. However, if the Examiner believes that any of the above-incorporated material constitutes “essential material” within the meaning of 37 CFR 1.57(c)(1)-(3), Applicants will amend the specification to expressly recite the essential material that is incorporated by reference as allowed by the applicable rules.

### SUMMARY

Aspects and applications of the invention presented here are described below in the drawings and detailed description of the invention. Unless specifically noted, it is intended that the words and phrases in the specification and the claims be given their plain, ordinary, and accustomed meaning to those of ordinary skill in the applicable arts. The inventor is fully aware that he can be his own lexicographer if desired. The inventor expressly elects, as his own lexicographer, to use only the plain and ordinary meaning of terms in the specification and claims unless he clearly states otherwise and then further, expressly sets forth the “special” definition of that term and explains how it differs from the plain and ordinary meaning. Absent such clear statements of intent to apply a “special” definition, it is the inventor's intent and desire that the simple, plain and ordinary meaning to the terms be applied to the interpretation of the specification and claims.

The inventor is also aware of the normal precepts of English grammar. Thus, if a noun, term, or phrase is intended to be further characterized, specified, or narrowed in some way, then such noun, term, or phrase will expressly include additional adjectives, descriptive terms, or other modifiers in accordance with the normal precepts of English grammar. Absent the use of such adjectives, descriptive terms, or modifiers, it is the intent that such nouns, terms, or phrases be given their plain, and ordinary English meaning to those skilled in the applicable arts as set forth above.

Further, the inventor is fully informed of the standards and application of the special provisions of 35 U.S.C. § 112(f). Thus, the use of the words “function,” “means” or “step” in the Detailed Description or Description of the Drawings or claims is not intended to somehow indicate a desire to invoke the special provisions of 35 U.S.C. § 112(f), to define the invention. To the contrary, if the provisions of 35 U.S.C. § 112(f) are sought to be invoked to define the inventions, the claims will specifically and expressly state the exact phrases “means for” or “step for, and will also recite the word “function” (i.e., will state “means for performing the function of [insert function]”), without also reciting in such phrases any structure, material or act in support of the function. Thus, even when the claims recite a “means for performing the function of . . . ” or “step for performing the function of . . . ,” if the claims also recite any structure, material or acts in support of that means or step, or that perform the recited function, then it is the clear intention of the inventor not to invoke the provisions of 35 U.S.C. § 112(f). Moreover, even if the provisions of 35 U.S.C. § 112(f) are invoked to define the claimed inventions, it is intended that the inventions not be limited only to the specific structure, material or acts that are described in the preferred embodiments, but in addition, include any and all structures, materials or acts that perform the claimed function as described in alternative embodiments or forms of the invention, or that are well known present or later-developed, equivalent structures, material or acts for performing the claimed function.

It may be a goal of the invention to be a secured medication dispensing device designed to force patient compliance with a prescribed medication schedule. This is sometimes accomplished through medication dispensers that remind patients to take their medication. These systems often allow the patient access to the full prescription at any given time. This invention, however, will not allow the patient to load the dispenser or have access to the full prescription at one time. Instead, this invention may be loaded by a pharmacist or other medical care provider and may limit access to only one dose or after a prescribed minimum time since the last taken dose. One non-limiting implementation is as follows: Dose 2 may be available 6 hours after Dose 1 is taken; Dose 3 may be available 6 hours after Dose 2 is taken; and Dose 4 may be available 6 hours after Dose 3 is taken; et cetera.

It may be another goal of the invention to be a secured medication dispensing device designed to minimize the risk of substance use disorder and/or other forms of substance addiction. This is generally accomplished through repeat medical provider visits and/or patient accountability, but a goal of this invention is to require neither. This invention will force patient compliance outside of a medical setting with an option for dosage tapering through flexible programing of minimum times between the availability of doses. One non-limiting implementation is as follows: Dose 2 may be available 6 hours after Dose 1 is taken; Dose 3 may be available 10 hours after Dose 2 is taken; and Dose 4 may be available 16 hours after Dose 3 is taken; et cetera.

It may be a goal of the invention to provide separated compartments in which a dose or doses of medication may be housed.

It may be a goal of the invention to force compliance with prescribed minimum times between doses through a lock. In one non-limiting embodiment of the lock, the lock may include a stem that may prevent rotation of components of the medication dispenser until a prescribed time interval has passed. Once the prescribed time interval has elapsed, the lock map unlock to allow rotation of components of the medication dispenser align a compartment within the base with the notch within the cap.

It may be another goal of the invention to be a secured medication dispensing device designed to prevent patient overdose of prescribed medication. Generally, a patient has access to their prescribed medication for an unlimited time after the window for which the medicine was prescribed has passed. Current devices that attempt to force patient compliance with a prescription schedule still allow a patient to remove all doses of the prescribed medication, regardless of the length of time since the medication schedule was prescribed to begin. A goal of this invention may be to force compliance through preventing access to any doses after a prescribed amount of time after the medication scheduled is set to begin or after a first dose of medication is accessed by a patient.

It may be another goal of the invention to be a secured medication dispensing device designed to prevent theft or improper transfer of prescribed medication between individuals and contamination of the public water supply. Many patients maintain untaken doses of medication, which, if accessible, can be taken without medical provider supervision by individuals other than the patient. Many other patients dispose of their prescribed medication down drains, contaminating the public water supply. A goal of this invention may be to lock all unused medication within the device, after which, the patient may return the device and unused medication to a pharmacist or other medical care provider for proper disposal.

It may be another goal of the invention to relieve concerns and causes of medical provider liability related to insufficient care. Medical providers may be concerned that the rigidity of an electronically regulated to medication dispenser may not account for the needs of patients who require momentary increases in medication doses. A goal of the invention may be to provide access to one or more doses of medication within continuously unlocked compartments that allow for a patient to receive potentially necessary dose(s) of medication outside of the prescription schedule, with effectiveness that may last long enough for the patient to contact the medical provider for a longer-term solution. In one non-limiting embodiment, these extra doses may be housed in a compartment within the cap of the medication dispensing device.

Moreover, it may be another goal of the invention to relieve concerns and causes of medical provider liability from cross contamination of medication. Because it may be a goal of the invention to be reused, and because the invention may house different types of medication in subsequent uses, it may be a goal of the invention to prevent cross-contamination between uses by providing a disposable, potentially sterile insert for each use of the invention. Non-limiting embodiments of this insert include trays without a cap, trays with a cap, bubble packs that may be pre-loaded with medication, and other inserts configured to fit within the medication dispenser.

It may be a goal of the invention to receive data, including, but not limited to, mediation type, patient information, medical provider information, prescribed minimum time between doses, prescribed maximum time during which any doses are accessible by the patient, and/or other data. In one non-limiting embodiment, this data may be transmitted to the medication dispensing device via a data port. In another non-limiting embodiment, this data may be transmitted via Bluetooth® or other wireless methods of information transmission.

It may be a goal of the invention to increase the likelihood that only the patient for whom the medication is prescribed has access to the medication within the device. In one non-limiting embodiment, this may be achieved through a lock. Non-limiting embodiments of this lock may include a lock paired with a key, a combination lock, electronic code lock, or other type of lock. Other non-limiting embodiments of this lock may include locks that verify patient identity, including, but not limited to, a fingerprint scanner, eye scanner, or other type of locks that verify an individual's identity.

The invention may require a power source. In one non-limiting embodiment, this power source may include one or more disposable batteries. In another non-limiting embodiment, this power source may include one or more rechargeable batteries. In embodiments that may use one or more rechargeable batteries, these batteries may be recharged externally from the device or while the batteries remain within the device.

One non-limiting embodiment of the invention may include a display screen. This display screen may display information, including, but not limited to mediation type, patient information, medical provider information, prescribed minimum time between doses, prescribed maximum time in which doses are accessible by the patient, battery life remaining, and/or other information.

One non-limiting embodiment of the invention may include a global positioning system or tracker.

One non-limiting embodiment of the invention may include a system that alerts a pharmacist or other medical provider of an attempt to tamper, break, disturb, or otherwise act in non-compliance with the device.

The foregoing and other aspects, features, and advantages will be apparent to those artisans of ordinary skill in the art from the DETAILED DESCRIPTION and DRAWINGS, and from the CLAIMS.

### BRIEF DESCRIPTION OF THE SEVERAL VIEWS OF THE DRAWINGS

A more complete understanding of the present invention may be derived by referring to the detailed description when considered in connection with the following illustrative figures. In the figures, like reference numbers refer to like elements or acts throughout the figures.

FIG. 1 depicts an isometric view of the invention when assembled.

FIG. 2 depicts an isometric view of the invention when disassembled.

FIG. 3 depicts an isometric view of the invention when assembled with a compartment in the cap of the invention.

FIG. 4 depicts an isometric view of the invention when assembled with a second lock in the cap of the invention.

FIG. 5 depicts an isometric view of the invention when disassembled with an insert between the cap and base of the invention.

Elements and acts in the figures are illustrated for simplicity and have not necessarily been rendered according to any particular sequence or embodiment.

### DETAILED DESCRIPTION

In the following description, and for the purposes of explanation, numerous specific details are set forth in order to provide a thorough understanding of the various aspects of the invention. However, it will be understood by those skilled in the relevant arts that the present invention may be practiced without these specific details. In other instances, known structures and devices are discussed more generally in order to avoid obscuring the invention. In many cases, a description of the operation is sufficient to enable one to implement the various forms of the invention. It should be noted that there are many different and alternative configurations, devices, and technologies to which the disclosed inventions may be applied. The full scope of the inventions is not limited to the examples that are described below.

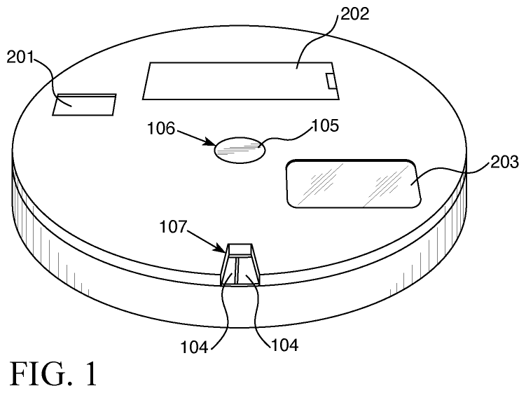

FIGS. 1 through 5 show a device in the form of a capsule designed for the controlled dispensing of medication.

This device may comprise a cap 101 removably and rotatably coupled to a base 102. The cap 101 may comprises a channel 106 at about a center-most point 106b of the cap 101 and a notch 107 in an outer edge 107b of the cap 101. The base 102 may comprise a rotation stem 105 protruding from about a center-most point 105B of the base 102. In the depicted embodiment, to assemble the device, the rotation stem 105 passes through the channel 106 within the cap, locking the two cap 101 and the base 102 together. To open the device for loading of medication, the cap 101 and the base 102 may be separated by detaching the rotation stem 105 from the channel 106 in the cap 101.

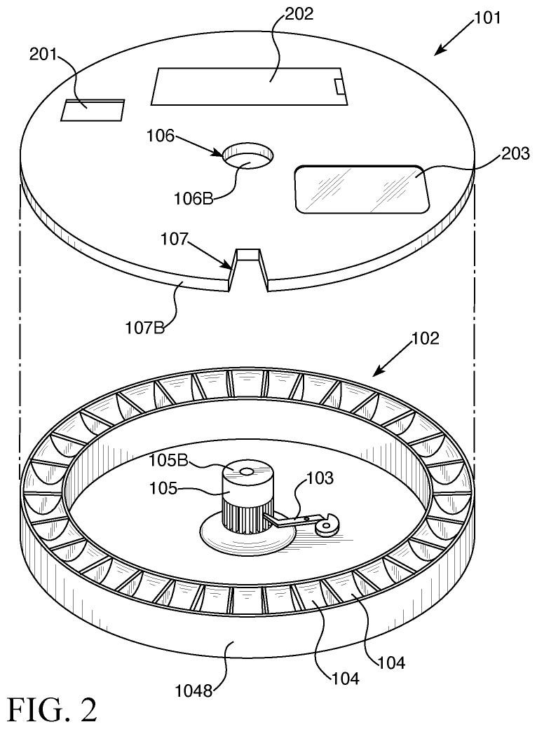

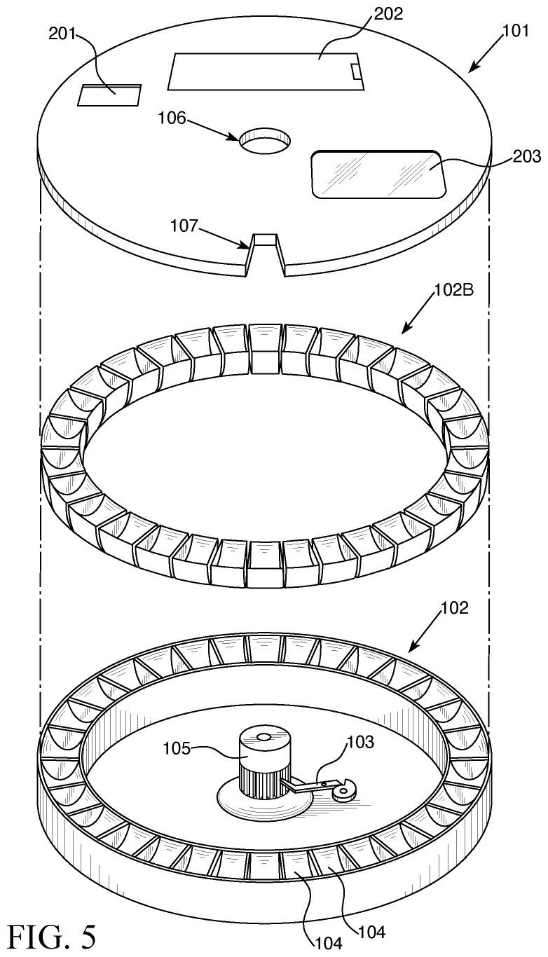

The device may further comprise a plurality of compartments 104 proximal to an outer edge of the base 1048 configured to house medication. As shown in FIGS. 2 and 5, the base 102 may contain thirty (30) compartments 104. The base 102, however, may contain any number of compartments 104 greater than one (1). These compartments 104 are to be loaded with a dose of medication by a medical provider. These compartments 104 are most easily accessible when the cap 101 and the base 102 are uncoupled, leaving all compartments 104 accessible at one time, as depicted in FIGS. 2 and 5. Once the medication is properly loaded within the compartments 104, the cap 101 and base 102 may be recoupled. The notch 107 within the cap 101 may be configured to facilitate ease of access to medication within the particular compartment 104 from which the patient has access at a particular time.

The device may further comprise a first lock 103 that prevents rotation of the cap 101 and the base 102 when locked. When unlocked, this lock 103 is designed to limit directionally opposite rotation of the cap 101 and the base 102 to prescribed ranges of degrees that correspond with the size of one ore more compartments 104. For example, if a medical provider prescribes for a patient to have access to only one dose of medication at time, the lock 103, when unlocked, will limit rotation of the cap 101 and the base 102 so that only a single compartment 104, and the medication within that compartment 104, are visible within and accessible via the notch 107 within the cap 101.

As shown in FIGS. 1 through 5, this capsule may be substantially cylindrical. The capsule, however, may be configured in any way that allows for the cap 101 and the base 102 to be removably and rotatably coupled in a manner that allows a patient to access medication through the alignment of the compartments 104 and the notch 107 in the cap.

The device may comprise a first timing system, wherein the first timing system unlocks the first lock 103 only after a prescribed time interval since a previous rotation of the cap 101 and the base 102. Once the patient rotates that the cap 101 and the base 102 to access a dose of medication, the lock 103 will relock. The first timing system will then begin counting down until the prescribed time interval has again elapsed, signaling the lock 103 to unlock.

The prescribed minimum time interval between doses regulated by the first timing system may be uniform or varied. One non-limiting embodiment of uniform minimum times between doses is as follows: Dose 2 may be available 6 hours after Dose 1 is taken; Dose 3 may be available 6 hours after Dose 2 is taken; and Dose 4 may be available 6 hours after Dose 3 is taken; et cetera. One non-limiting implementation of varied minimum time intervals between doses is as follows: Dose 2 may be available 6 hours after Dose 1 is taken; Dose 3 may be available 10 hours after Dose 2 is taken; and Dose 4 may be available 16 hours after Dose 3 is taken; et cetera.

The device may further comprise a second timing system, wherein the second timing system prevents the first timing system from unlocking the lock 103 after a prescribed time interval. A medical provider can prescribe for the second timing system to begin counting down at a specific time or to begin when the patient first rotates the cap 101 and the base 102 to access a dose of medication. Once initiated, the second timing system counts down independent of the first timing system. Once the prescribed time interval within the second timing system has elapsed, the second timing system overrides the first timing system's ability to unlock the lock 103.

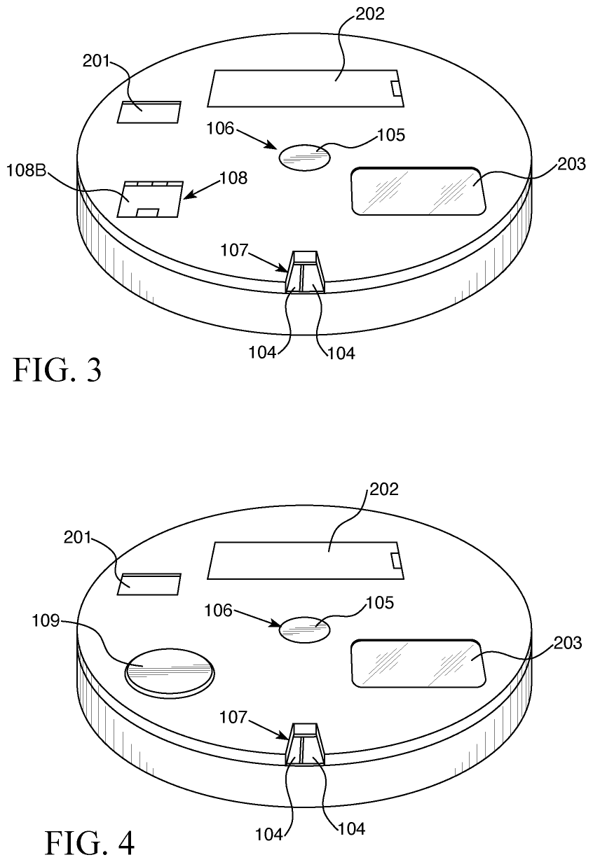

The device may further comprise, as depicted in FIG. 3, a compartment 108 and a removably coupled lid 108B. This compartment 108 may be configured to house one or more doses of medication. The lid 108B of this compartment 108 is to remain unlocked at all times. The purpose of this compartment 108 is to provide access to doses of medication outside of the medication scheduled articulated by the first timing system and the second timing system.

The device may further comprise, as depicted in FIG. 4, a second lock 109 that prevents rotation of the cap 101 and the base 102 when locked. This second lock 109 may be used to increase the likelihood that only the patient for whom the medication within the device is prescribed has access to the medication.

The device may further comprise, as depicted in FIG. 5, an insert 102B configured to removably couple to the plurality of compartments 104 proximal to the outer edge 104B of the base 102. This insert 102B may be used to prevent cross contamination of different medications housed within the compartments 104 of the device on subsequent uses of the device. This insert 102B may also be preloaded with medication in a bubble pack or other similar structure.

FIGS. 1 through 5 depict the device comprising a data port 201 to receive data into the device. The data port 201 may be used to receive information including, but not limited to, mediation type, patient information, medical provider information, prescribed minimum time between doses, prescribed maximum time during which any doses are accessible by the patient, and/or other data. The data port 201 may not be necessary in all embodiments of the device, as the previously described information may also be transmitted to the device via wireless communication.

FIGS. 1 through 5 depict the device with a battery compartment 202. The battery compartment 202 is used to house the power source for the device. The batteries housed within the battery compartment 202 may be disposable and/or rechargeable batteries. In embodiments that use rechargeable batteries, these batteries may be recharged externally from the device or while the batteries remain within the device.

FIGS. 1 through 5 depict the device with a display screen 203. This display screen 203 may include information, including, but not limited to mediation type, patient information, medical provider information, prescribed minimum time between doses, prescribed maximum time in which doses are accessible by the patient, battery life remaining, and/or other information.

The device may comprise any combination of the above described elements. For example, one embodiment of the device may include the compartment 108 and removably coupled lid 108B depicted in FIG. 3, the second lock 109 depicted in FIG. 4, and/or the insert 102B depicted in FIG. 5.

Many of the components of the embodiments described above may be commercially available. Those components that are not commercially available may be manufactured using a variety of materials suitable for their intended purposes, including but not limited to various metals, plastics, and fabrics. The cap 101, base 102, insert 102B, first lock 103, compartments 104, outer edge of the base 1048, rotation stem 105, outer edge of the cap 107B, compartment within the cap 108, lid to the compartment within the cap 108B, second lock 109, data port 201, battery compartment 202, and display screen 203 may be made of metal, plastic, glass, plexiglass, or other rigid materials.

Inventions pertaining to a device and method for controlled dispensing of medication are described above. Various changes may be made to the inventions without departing from their scope. The above descriptions of non-limiting, exemplary embodiments are provided for the purpose of illustration only and not limitation, with the invention being defined by the claims and equivalents thereof.

1. A medication dispenser, comprising:
a capsule further comprising a cap removably and rotatably coupled to a base, wherein the cap comprises a channel at about a center-most point of the cap and a notch in an outer edge of the cap, and the base comprises a rotation stem protruding from about a center-most point of the base;
a plurality of compartments proximal to an outer edge of the base configured to house medication;
a first lock, wherein the first lock prevents rotation of the cap and the base when locked and limits directionally opposite rotation of the cap and the base to a prescribed range of degrees when unlocked;
a first timing system, wherein the first timing system unlocks the first lock only after a first prescribed time interval has elapsed, the first prescribed time interval comprising a minimum amount of time since a previous rotation of the cap and the base; and
a second timing system, wherein the second timing system prevents the first timing system from unlocking the first lock after a second prescribed time interval has elapsed.

2. The medication dispenser of claim 1, wherein the second timing system is initiated by a first rotation of the cap and the base.

3. The medication dispenser of claim 1, wherein the cap of the capsule further comprises a compartment and a removably coupled lid.

4. The medication dispenser of claim 1, further comprising a second lock that prevents rotation of the cap and the base when locked.

5. The medication dispenser of claim 1, further comprising an insert configured to removably couple to the plurality of compartments proximal to the outer edge of the base.

6. The medication dispenser of claim 1, wherein the first prescribed time interval remains uniform in length throughout the second prescribed time interval.

7. The medication dispenser of claim 1, wherein the first prescribed time interval changes in length at least once during the second prescribed time interval.

8. The medication dispenser of claim 7, wherein the first prescribed time interval progressively increases in length during the second prescribed time interval.

9. A method for controlling medication access, comprising:
providing a medication dispenser comprising a cap that is removably and rotatably coupled to a base, wherein the base comprises a plurality of compartments proximal to an outer edge of the base configured to house medication;
locking a first lock within the medication dispenser;
unlocking the first lock according to a first prescribed time interval, wherein the first prescribed time interval comprises a length time between when a patient accesses a dose of medication within the medication dispenser and when the patient is permitted to access a subsequent dose of medication within the medication dispenser; and
terminally locking the first lock according to a second prescribed time interval.

10. The method for controlling medication access in claim 9, wherein the second prescribed time interval comprises an amount of time from when the patient accesses a first dose of medication within the medication dispenser.

11. The method for controlling medication access in claim 9, wherein the first prescribed time interval remains uniform in length throughout the second prescribed time interval.

12. The method for controlling medication access in claim 9, wherein the first prescribed time interval changes in length at least once during the second prescribed time interval.

13. The method for controlling medication access in claim 12, wherein the first prescribed time interval progressively increases in length during the second prescribed time interval.

14. The method for controlling medication access in claim 9, further comprising developing a return program, wherein the medication dispenser is returned to a provider of the medication dispenser after the termination of the second prescribed time interval.

15. The method for controlling medication access in claim 9, further comprising providing an insert configured to removably couple to the plurality of compartments proximal to the outer edge of the base.
